(19) United States
(12) Patent Application Publication
(10) Pub. No.: US 2005/0043820 A1
(43) Pub. Date: Feb. 24, 2005

(54) Biomaterial comprising microfeatures

(76) Inventor: James Browning, Glasgow (GB)

Correspondence Address: Gregory J Lavorgna;Drinker Biddle & Reath, One Logan Square, 18th & Cherry Streets, Philadelphia, PA, 19103-6996, US

(21) Appl. No.: 10/492,473

(22) PCT Filed: Oct. 14, 2002
(86) PCT No.: PCT/GB02/04661

Related U.S. Application Data
(60) Provisional application No. 60/394,114, filed on Jul. 5, 2002.

(30) Foreign Application Priority Data
Oct. 12, 2001 (GB) 0124489.6

(51) Int. Cl. A61F 2/02

(52) U.S. Cl. 623/23.74; 606/151; 600/37

(57) ABSTRACT

The present invention relates to biomaterials for use in surgical implants. The biomaterial can include micropatterns; such as microgrooves and microfeatures such as pores, pits or slits on the surface of the biomaterial. The micropattern can be parallel microgrooves capable of influencing the orientation and alignment of cells proliferating on the surface of the biomaterial. The microfeatures enable tissue ingrowth into the biomaterial and may extend through all or part of the biomaterial, allowing the rapid integration of the implant into the tissue.

The present invention relates to materials for use in the manufacture of surgical devices, in particular the present invention provides improved biomaterials for use in the manufacture of surgical implants.

Many surgical procedures involve the introduction of implants into the human or animal body to repair or replace defective body walls, support organs, tissues of the body or replace parts of the body such as valves which are defective.

To date these implants have consisted of the transplant of autologous, homologous or heterologous biological material such as skin (dermis) or muscle.

Artificial implants which have been typically comprised of synthetic material such as metallic and carbon fibre meshes. Implants formed from polymeric meshes are also known.

The synthetic materials currently used in the manufacture of surgical implants exhibit a wide range of properties. Different materials are therefore used for particular purposes with the aim of achieving optimal performance.

Metal and carbon fibre meshes have been shown to become work-hardened, inflexible, friable and fragmented in time. Further, implants consisting of metal or carbon fibre material have been observed to protrude through skin or body walls or erode into adjacent tissue or blood vessels.

A number of synthetic polymeric meshes comprised of Dacron™ (Mersilene™), polypropylene (Marlex™ and Prolene™) and Teflon™ have been used to manufacture surgical implants. These synthetic surgical implants do not suffer from the disadvantages discussed above of metal and carbon fibre. Further, surgical implants comprised of polymeric mesh material are suitably inert and as such are less likely to degrade or cause an adverse reaction. These synthetic polymeric meshes are also mechanically strong, cheap and easily sterilisable.

A disadvantage of synthetic polymeric meshes is that they are relatively rigid. This leads to problems in placing the meshes within the body. In addition these polymeric meshes typically have rough surfaces, which although once the implants have been positioned aid the retention of the implant in the body, cause difficulty during the initial positioning of the implant in the desired location.

Erosion of adjacent tissues by the mesh implants, or rough edges of the implant can also lead to the development of fistula or sinus, abnormal passages between internal organs or between internal organs and the body surface.

One surgical procedure in which a surgical implant is placed in the body is that used to treat female urinary incontinence. In this procedure a sling of tape material is passed under the urethra such that it is positioned loosely under the urethra and supports the urethra, with a supporting member being suspended between two members such that the supporting member forms a sling under the urethra.

Typically the sling tape members are comprised of synthetic mesh such as knitted polypropylene (Prolene™). The sling members are initially placed in position by the surgeon and are subsequently held in place by the rough edges of the sling members via friction between the rough edges of the sling and the surrounding body tissue. The surrounding body tissue then grows around the sling members over a period of time securing them to the tissue and holding the supporting member in place.

A number of disadvantages of using the sling mesh members presently used in the above procedure exist.

One disadvantage is that the surgical implants presently used in such procedures do not include tissue engineering features on the surface of the implant and therefore the laydown of collagen on the implant surface is chaotic. The lack of tissue engineering features on the sling means that no axial alignment of the fibroblasts proliferating of the surface of the sling mesh members occurs, which results in decreased strength of the tissues surrounding the sling mesh members. Due to the lack of mechanical strength of the non-axially aligned fibroblasts, in order to be effective, the implanted surgical implant must remain in place in the body to provide the mechanical strength to support the urethra for a long period of time.

A further disadvantage of the sling mesh members currently used in the TVT procedure is that tissue ingrowth into the material is very slow. The pores present in the material which typically forms the sling mesh members are not designed to aid tissue ingrowth, and pores present in material used to form such mesh members are too large to promote efficient in-growth of fibroblasts into the material. Typically the pores of the mesh material used in the prior art are of dimension 600-1700 μm. The lack of microfeatures to provide and enable tissue ingrowth means that the implant takes a long time to be incorporated into the surrounding tissues. This requires the implant to be of greater mass in order to be of suitable strength and provide suitable support in the body.

Further the sling mesh members described in the prior art consist of substantial mass. Ideally in order to decrease the risk of inflammation and other complications, as little foreign material should be implanted into the patient as possible.

In view of the disadvantages associated with known material used to manufacture surgical implants for human and animal bodies it would be advantageous to provide a new biomaterial or modify an existing material such that the material showed improved characteristics for use in a surgical implant.

According to a first aspect of the present invention there is provided a biomaterial, having at least one micropattern on at least one surface of the biomaterial, the micropattern including a plurality of substantially parallel grooves said grooves being capable of influencing the orientation and alignment of cells proliferating on the surface of the biomaterial.

The biomaterial may be synthetic, non absorbable or absorbable and/or biological.

Non absorbable materials may be preferred where the implant is required to provide additional tissue support. However, absorbable materials which absorb slowly, i.e. between 6 to 12 months after implantation can usefully influence cell proliferation at the requested time, providing support when required before supporting tissue has formed.

The dimensions of the grooves are such that they are under the size of a typical cell body. For example fibroblasts are typically 20 to 30 μm in diameter and therefore grooves to influence the orientation and alignment of fibroblasts can be up to 20 μm wide.

Preferably the grooves are 0.5 to 20 μm in width and 0.25 to 20 μm in depth.

More preferably the grooves are 4 to 6 μm in width and 4 to 6 μm in depth.

In a particular embodiment the grooves are 5 μm in width and 5 μm in depth.

Preferably the grooves are separated by ridges of between 1.0 to 20 μm in width.

More preferably the grooves are separated by ridges 4 to 6 μm in width.

In a particular embodiment the grooves are separated by ridges of 5 μm in width.

Preferably the grooves present on the surface of the biomaterial are aligned in the same direction.

Alternatively the grooves are arranged in groups with the grooves in a particular group being aligned in a similar direction, and different groups of grooves being aligned in different directions.

Preferably the ridges are formed by square pillars and the base of the microgroove is substantially perpendicular to the side walls of the square pillars.

Alternatively the ridges are formed by square pillars and the base of the microgrooves is bevelled in relation to the side walls of the square pillars.

Alternatively the side walls of the pillars may be arcuate.

Preferably the grooves extend along the length of at least one surface of the biomaterial.

More preferably the grooves extend along a first surface and a second opposite surface of the biomaterial.

Alternatively the grooves are only present in a defined area of the biomaterial.

Preferably the biomaterial is between 50cm and 300Am thick.

More preferably the biomaterial is between 100 to 250 μm thick.

In a particularly preferred embodiment the biomaterial is 200 μm thick.

According to a second aspect of the present invention there is provided a synthetic biomaterial including at least one microfeature which promotes tissue ingrowth through the biomaterial.

In one embodiment the microfeature comprises at least one pore which extends through the biomaterial from a first surface of the biomaterial to a second opposite surface of the biomaterial said pore ranging in width across the surface of the biomaterial from 50 μm to 300 μm.

In a second embodiment the microfeature comprises at least one pit which indents but does not extend through the biomaterial said pit ranging in width across the surface of the biomaterial from 50 μm to 300 μm.

In a third embodiment the microfeature comprises at least one slit which extends through the biomaterial from a first surface of the biomaterial to a second opposite surface in the biomaterial wherein said slit is from 50 μm to 2 mm in length and from 50 μm to 500 μm in width.

Preferably the biomaterial comprises at least one slit of length from 50 μm to 1 mm and width 100 μm.

More preferably the biomaterial comprises at least one slit of length 200 μm and width 50 μm.

The slits of the biomaterial may be orientated such that their longest dimension is parallel to the longitudinal axis of the biomaterial.

Alternatively the slits of the biomaterial may be orientated such that their longest dimension is not parallel to the longitudinal axis of the implant.

Preferably the biomaterial comprises pits or pores ranging in width across the biomaterial from 100-150 μm.

In one embodiment the microfeatures are distributed across the complete surface of the biomaterial.

Alternatively microfeatures are distributed only in a particular portion of the surface of the biomaterial.

Preferably microfeatures are created by post synthesis modification of synthetic biomaterial.

Preferably microfeatures are created by post synthesis treatment of the surface of synthetic biomaterial by a laser.

More preferaby microfeatures are created by post synthesis treatment of the grooved surface of synthetic biomaterial by a laser.

Alternatively microfeatures of between 50-200 μm in width are created during synthesis of the synthetic biomaterial.

Preferably microfeatures formed during the synthesis of synthetic material are formed by spaces between the waft and weave of mono-filament or multi-filament yarns when they are woven to form a mesh.

Alternatively microfeatures formed during the synthesis of synthetic material are formed by the inter-filament spaces created when mono-filaments are twisted to create multi-filaments, the multi-filaments then being woven to form a mesh.

The biomaterial may be a material that is not absorbed into the surrounding tissues over time.

Alternatively the biomaterial may be absorbable into the surrounding tissues over time.

Preferably the biomaterial is absorbable into the surrounding tissues in less than 12 to 18 months following insertion of the biomaterial into the body.

More preferably the biomaterial is absorbable into the surrounding tissue in less than 10 to 12 months following the insertion of the biomaterial into the body

obviously the choice of biomaterial will depend on the application.

Preferably the biomaterial comprising at least one micropattern further includes at least one microfeature, wherein the microfeature includes at least one pore, pit or slit.

The microfeatures in the biomaterial comprising pores, pits or slits allow fibroblastic through growth of the biomaterial to allow fixation of the biomaterial in the tissue. The fixation provides multilevel adhesion of the biomaterial to the tissue along its length.

An embodiment of the present invention will now be described by way of example only with reference to the accompanying drawings in which,

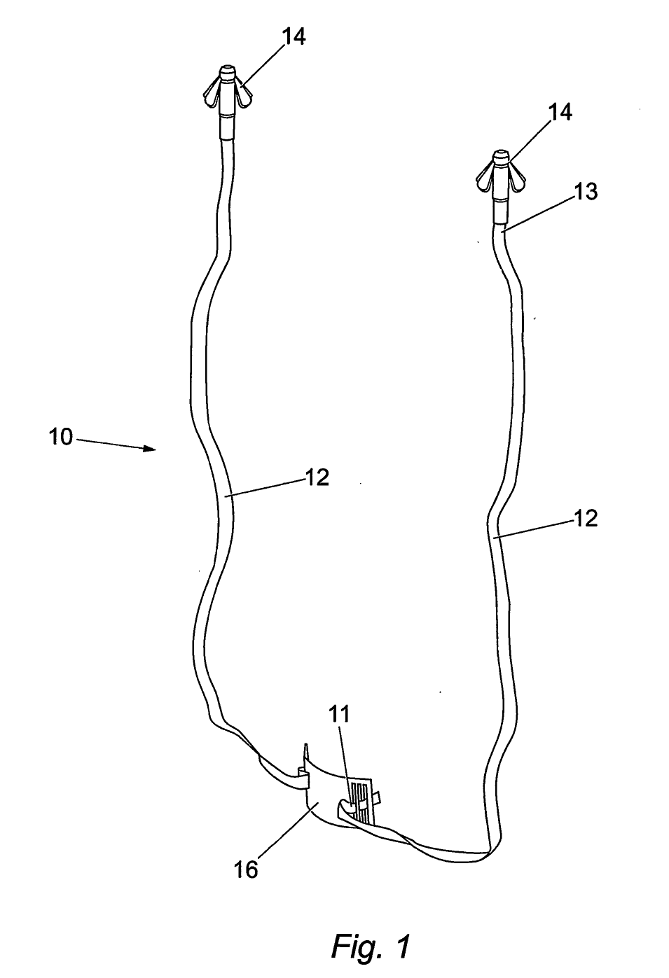

FIG. 1 shows an illustration of a surgical implant utilising the biomaterial,

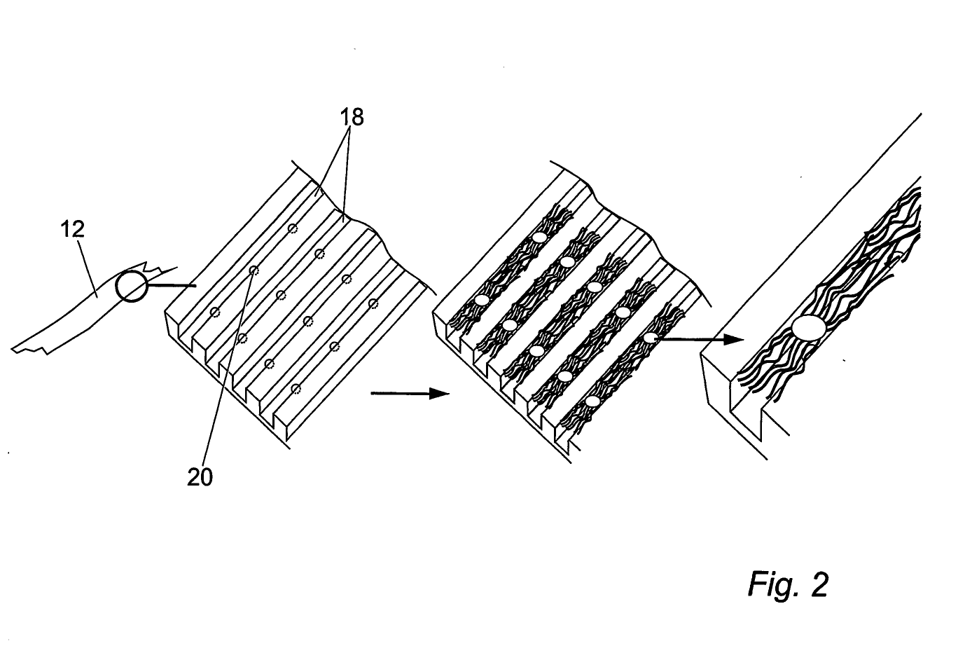

FIG. 2 shows an illustration of the biomaterial comprising grooves and pores.

As shown in FIG. 1 the biomaterial can be utilised to form a surgical implant device 10 to support the urethra and alleviate urinary stress incontinence. The surgical implant device 10 comprises two tape members 12 each tape member being connected at a first end 11 to a support member 16 such that the support member lies between the each tape member. Tissue anchor members 14 are conjoined to the tape members 12 at a second end 13 opposite the first end 11 of each tape member 12.

As shown in FIG. 2 a micropattern in the form of parallel microgrooves 18 of a size in the range 1-7 μm wide and 0.45-7.0 μm deep and separated by ridges 1-5 μm in width extends along the surface of the tape members 12. In the example shown, the micropattern is orientated such that the grooves extend from the first end 11 of each tape member 12 attached to the suburethral support 16 to the second end 13 of each tape member 12 conjoined to the tissue anchor 14.

In addition to, or independently to, the presence of the micropatterns on the surface of the tape members 12 the tape members 12 may also comprise microfeatures to promote tissue ingrowth. The microfeatures may comprise pores 20 in the range of 50-200 μm in size, which aid the incorporation of fibroplasia into meshes of the surgical implant.

The microfeatures may also comprise pits which indent the surface of the biomaterial or slits in the biomaterial. The biomaterial can consist of only one type of microfeature, for example pores, or may comprise a range of different microfeatures including pores, pits and slits.

In use the suburethral support 16 is positioned loosely under the urethra by a surgeon during a procedure to insert the surgical implant device 10 into the body. The suburethral support 16 being held in place by the tape members 12, which are attached to the body via the tissue anchors 14. The suburethral support 16 supports the urethra and alleviates urinary incontinence by occluding the mid-urethra at times of raised abdominal pressure induced by coughing or the like.

As discussed above, sling members used to support the urethra and alleviate urinary stress incontinence of the prior art, are comprised of synthetic mesh such as knitted polypropylene (Prolene™) and following implantation of these prior art surgical implant devices, tissue growth occurs around the surgical implant, the proliferating cells on the surface of the surgical implant not being influenced by the surface of the surgical implant to adopt any particular orientation or alignment. This means that tissue with non optimal structure for support of the urethra and incorporation of the implant into the body is laid down.

The biomaterial of the present invention can be synthesised from a suitable polymer material as known in the art which is biologically-acceptable. One example of a suitable synthetic material is polycaprolactone, but it is understood that the skilled man would envisage other suitable polymers such as Prolene.

Alternatively natural materials or polymers can be used to form the biomaterial such natural polymers including collagen and polysaccharides. Where natural materials are used to form the biomaterial, micropatterns and microfeatures can be incorporated by suitable means. For instance a laser can be used to form the micropatterns and/or microfeatures. Alternatively mechanical means can be used to provide the micropatters and microfeatures.

Suitable material for use as a biomaterial would have to provide adequate tissue support. For example if the biomaterial is to be used as a sling member to support the urethra the material chosen to form the biomaterial would have to have sufficient strength to support the urethra at times of increased abdominal pressure. Similarly if the biomaterial is to be used to repair fascial defects or as surgical implants for use in treating hernia or vaginal prolapse the material forming the biomaterial would require to have suitable strength, pliability, and resilient characteristics.

Should the material used to form the biomaterial have absorbable characteristics, the absorbable material would have to remain in the body for a sufficient period of time in order to provide support for instance to the urethra until supporting tissue formed. It would be expected that the biomaterial should remain in the body for at least 6 months from implantation to allow suitable supporting tissue to form. In a particular embodiment the absorbable biomaterial would remain in the body for 12 months from implantation in the body.

Microgrooves present on the biomaterial of the present invention of between 0.5 to 20 μm in width and 0.25 to 20 μm in depth induce cell orientation and cellular alignment along the direction of the grooves such that cellular organisation of the proliferating cells is altered such that the new tissue laid down on the surface of the tape members 12 of the surgical implant show improved qualities of mechanical strength. The microgrooves can act to control cell orientation and the shape of the cells being laid down. As the orientation of cells being laid down is controlled by the microgrooves, the direction of cell division will be influenced, the surface of the implant can thus promote the laydown of collagen or types of cells and lead to the formation of a neoligament structure or the growth of other biological structural forms.

The dimensions of the grooves of the micropatterns are determined by the cells which are to be laid down on the implant. For instance particular widths and depths of microgrooving can be chosen to encourage the lay down and orientation of fibroblasts (typical cell body around 20-30 μm), muscle cells or epithelial cells or to minimise the lay down of particular cells such as inflammatory cells on the implant. In view of the above the width and depth of the grooves provided can be restricted within particular ranges such as, grooves with a width and depth of between 1 to 5 μm, grooves with a width and depth of between 6 to 10 μm, grooves with a width and depth of between 11 to 15 μm or grooves with a width and depth of between 15 to 20 μm, depending on the purpose of the groove.

In particular examples of the biomaterial, the grooves may be aligned such that they are substantially parallel to each other providing the grooved surface of biomaterial with a corrugated appearance at the micrometer scale.

The ordered laydown of cells on the surface of the biomaterial following the introduction of the material in the body, means that the surgical implant can be designed to cope with less mechanical stress. This enables the tape members 12 and support member 16 to be formed of material which contains less mass. As previously indicated this is of importance as it decreases the risk of inflammation around the implant following its introduction into the body. The presence of microgrooving on the surface of the tape members 12 is capable of orientating and aligning the cells such that cells proliferating on the surface of the surgical tape can have sufficient mechanical strength to support the urethra without the presence of the tape members over time, the tape members can thus be formed from biomaterial which is capable of being absorbed into the body with time.

The use of material which is completely absorbed to form the biomaterial disclosed herein to produce surgical implants is advantageous, as it reduces the chances of inflammation, infection; translocation and fistula around the surgical implant.

In addition to organising the orientation and alignment of cells proliferating on the surface of the surgical implants it would also be advantageous if tissue could be quickly incorporated into the surgical implant. Faster tissue incorporation into the surgical implants would not only more firmly hold the implant in place, but should speed up wound healing and reduce complications such as fistula. Faster tissue integration of the biomaterial and thus the surgical implant can be gained by providing the biomaterial with microfeatures which promote tissue ingrowth into the biomaterial. Suitable microfeatures includes suitably dimensioned pores, or slits which extend through the biomaterial from a first surface to a second opposite surface or pits that indent the surface of the biomaterial.

As shown in FIG. 1, pores 20 of dimension 50-200 μm can be incorporated into the material, which forms sling tape members. In the case of synthesised biomaterial these pores 20 may be incorporated into the material which forms the tape members either during the original synthesis of the material or during post synthesis modification of the material.

In the surgical device shown in FIG. 1 pores of 50-200 μm are created by post synthesis modification of the biomaterial in which the biomaterial is scanned by a laser capable of forming pores of a suitable size in the material. The pores formed may be of any shape and the perimeter of the pore may be either smooth or rough. In the surgical device shown in FIG. 1, tissue ingrowth is only wished for into the tape members 12 and thus no pores 20 are incorporated into the support member 16.

The use of pores between the size of 50-200 μm on the tape members means that fibrous tissue and vascularised connective tissue are able to grow through the tape members 12 to strongly attach the tape members 12 to the surrounding tissues.

The addition of pores in the size range 50-200 μm to materials currently used to manufacture surgical implants via post synthesis means such as lasering would significantly improve the through growth of tissues into these materials. The pore size of the meshes currently used in the manufacture of surgical implants such as Dacron™ (Prolene™), Teflon™ and polypropylene (Marlex™) created during synthesis of these polymeric meshes are significantly larger than 50-200 μm. The interfibre spaces or spaces between the interfibre networks typically being in the range of 1100 μm, 700 μm and 1700 μm for Mesilene, Marlex and Teflon respectively.

As an alternative to pores, slits may be incorporated into the biomaterial to allow tissue ingrowth. The slits may be in the range 50 μm to 2 mm in length and 50 μm to 500 μm in width. The actual dimensions of the slit produced in the fabric may be varied within these ranges to optimise both tissue ingrowth required and the ease of manufacture of the implant. The slit may be orientated in any preferred fashion in relation to the dimensions of the implant to achieve the desired through growth and/or ease of manufacture.

In a further embodiment of the present invention the biomaterial described herein may be used to form a surgical implant for treatment of Uterovaginal prolapse or other bodily hernia.

In particular the use of biomaterial as described herein to produce surgical implants including meshes or patches for use in vaginal prolapse or hernia operations and to remedy fascial defects can be envisaged. In each of these cases, minimal implant mass, tissue incorporation into the implant and ordered tissue laydown are preferable. The use of biomaterial which incorporates micropatterns and microfeatures suitable for the purpose of the implant would enable improved surgical implants to be provided for use in procedures relating to these problems.

The biomaterial can be used to form a patch which extends over the site of the fascial defect, strengthening the tissue around the fascial defect and providing a structure to retain any organs, or other bodily parts which are pushed through the fascial defect during times of increased pressure.

Further the biomaterial described herein may be used to form implants in other regions of the human or animal body which would benefit from through growth of tissues and/or the formation of neoligaments.

Various modifications can be made without departing from the scope of the invention for example, it is envisaged that where the biomaterial is synthesised the pores of the biomaterial described herein may be formed during synthesis of the biomaterial.

The pores may be created during the synthesis of the polymeric meshes by the interfibre spaces. Alternatively the pores may be created during the synthesis of the polymeric meshes by the spaces between the interfibre network.

In addition it is envisaged that specific regions rather than the complete area of the biomaterial may be synthesised or treated such that they comprise pores in the range 50-200 μm in size, or slits in the range, length 50 μm-2 mm, width 50 μm-500 μm.

1. A biomaterial comprising at least one micropattern on at least one surface of the biomaterial the micropattern including a plurality of substantially parallel grooves said grooves being capable of influencing the orientation and alignment of cells proliferating on the surface of the biomaterial.

2. A biomaterial as claimed in claim 1 wherein the grooves are 0.5 to 20 μm in width and 0.25 to 20 μm in depth.

3. A biomaterial as claimed in claim 1 wherein the grooves are 5 μm in width and 5 μm in depth.

4. A biomaterial as claimed in claim 1 wherein the grooves are separated by ridges of between 1.0 to 20 μm in width.

5. A biomaterial as claimed in claim 4 the grooves are separated by ridges of 5 μm in width.

6. A biomaterial as claimed in claim 1 wherein the grooves present on the surface of the biomaterial are aligned in the same direction.

7. A biomaterial as claimed in claim 1 wherein the grooves are arranged in groups with the grooves in a particular group being aligned in a similar direction, and different groups of grooves being aligned in different directions.

8. A biomaterial as claimed in claim 4 wherein the ridges are formed by square pillars and the base of the microgroove is substantially perpendicular to the side walls of the square pillars.

9. A biomaterial as claimed in claim 4 wherein the ridges are formed by square pillars and the base of the microgrooves is bevelled in relation to the side walls of the square pillars.

10. A biomaterial as claimed in claim 8 wherein the side walls of the pillars are arcuate.

11. A biomaterial as claimed in claim 1 wherein the grooves extend along the length of at least one surface of the biomaterial.

12. A biomaterial as claimed in claim 1 wherein the grooves extend along a first surface and a second opposite surface of the biomaterial.

13. A biomaterial as claimed in claim 1 wherein the grooves are only present in a defined area of the biomaterial.

14. A biomaterial as claimed in claim 1 wherein the biomaterial is between 50 μm and 300 μm thick.

15. A biomaterial as claimed in claim 1 wherein the biomaterial is 200 μm thick.

16. A biomaterial comprising at least one microfeature to promote tissue ingrowth wherein the microfeature is at least one pore which extends through the biomaterial from a first surface of the biomaterial to a second opposite surface of the biomaterial said pore ranging in width across the surface of the biomaterial from 50 μm to 300 μm.

17. A biomaterial comprising at least one microfeature to promote tissue ingrowth wherein the microfeature comprises at least one pit which indents but does not extend through the biomaterial said pit ranging in width across the surface of the biomaterial from 50 μm to 300 μm.

18. A biomaterial comprising at least one microfeature to promote tissue ingrowth wherein the microfeature comprises at least one slit which extends through the biomaterial from a first surface of the biomaterial to a second opposite surface in the biomaterial wherein said slit from 50 μm to 2 mm in length and from 50 μm to 500 μm in width.

19. A biomaterial as claimed in claim 18 wherein the slit is of length from 50 μm to 1 mm and width 100 μm.

20. A biomaterial as claimed in claim 18 wherein the slit is of length 200 μm and width 50 μm.

21. A biomaterial as claimed claim 18 wherein the slit is orientated such that the longest dimension is parallel to the longitudinal axis of the biomaterial.

22. A biomaterial as claimed in claim 18 wherein the slit is orientated such that the longest dimension is not parallel to the longitudinal axis of the implant.

23. A biomaterial as claimed in claim 17 to wherein the microfeature ranges in width across the biomaterial from 100-150 μm.

24. A biomaterial as claimed in claim 16 wherein microfeatures are distributed across the complete surface of the biomaterial.

25. A biomaterial as claimed in claim 13 wherein micro-features are distributed only in a particular portion of the surface of the biomaterial.

26. A biomaterial as claimed in claim 16 wherein microfeatures are created by post synthesis modification of synthetic biomaterial.

27. A biomaterial as claimed in claim 26 wherein microfeatures are created by post synthesis treatment of the surface of synthetic biomaterial by a laser.

28. A biomaterial as claimed in claim 16 wherein microfeatures are created during synthesis of the synthetic biomaterial.

29. A biomaterial as claimed in claim 28 wherein the microfeatures formed during the synthesis of synthetic biomaterial are formed by spaces between the waft and weave of mono-filament or multi-filament yarns when they are woven to form a mesh.

30. A biomaterial as claimed in claim 28 wherein the microfeatures formed during the synthesis of synthetic biomaterial are formed by the inter-filament spaces created when mono-filaments are twisted to create multi-filaments, the multi-filaments then being woven to form a mesh.

31. A biomaterial as claimed in claim 1 wherein the biomaterial is not absorbed into the surrounding tissues over time.

32. A biomaterial as claimed in claim 1 wherein the biomaterial is absorbable into the surrounding tissues over time.

33. A biomaterial as claimed in claim 32 wherein the biomaterial is absorbable into the surrounding tissues in less than 12 to 18 months following insertion of the biomaterial into the body.

34. A biomaterial as claimed in claim 32 wherein the biomaterial is absorbable into the surrounding tissue in less than 10 to 12 months following the insertion of the biomaterial into the body.

35. A biomaterial as claimed in claim 1 which further comprises at least one microfeature to promote tissue ingrowth wherein the microfeature is at least one pore which extends through the biomaterial from a first surface of the biomaterial to a second opposite surface of the biomaterial said pore ranging in width across the surface of the biomaterial from 50 μm to 300 μm.

36. A biomaterial as claimed in claim 1 which further comprises at least one microfeature to promote tissue ingrowth wherein the microfeature comprises at least one pit which indents but does not extend through the biomaterial said pit ranging in width across the surface of the biomaterial from 50 μm to 300 μm.

37. A biomaterial as claimed in claim 1 which further comprises at least one microfeature to promote tissue ingrowth wherein the microfeature comprises at least one slit which extends through the biomaterial from a first surface of the biomaterial to a second opposite surface in the biomaterial wherein said slit from 50 μm to 2 mm in length and from 50 μm to 500 μm in width.

38-40. (Cancelled)
